(19) United States
(12) Patent Application Publication
(10) Pub. No.: US 2025/0221764 A1
(43) Pub. Date: Jul. 10, 2025

(54) HYBRID MAPPING AND PULSED FIELD ABLATION CATHETER

(71) Applicant: St. Jude Medical, Cardiology Division, Inc., St. Paul, MN (US)

(72) Inventors: Greg OLSON, Elk River, MN (US); Isaac REMER, Richfield, MN (US)

(21) Appl. No.: 18/853,575

(22) PCT Filed: Apr. 4, 2023
(86) PCT No.: PCT/US2023/065321

§ 371 Date: Oct. 2, 2024

Related U.S. Application Data
(60) Provisional application No. 63/327,908, filed on Apr. 6, 2022.

(51) Int. Cl. A61B 18/14; A61B 18/00

(52) U.S. Cl. CPC A61B 18/1492; A61B 2018/00071; A61B 2018/00077; A61B 2018/00178; A61B 2018/00184; A61B 2018/00267; A61B 2018/00351; A61B 2018/00577; A61B 2018/00613; A61B 2018/00702; A61B 2018/00839; A61B 2018/1467; A61B 2218/002

(57) ABSTRACT

Embodiments of the present disclosure describe hybrid catheters having flexible array of electrodes configured for both mapping and high power ablation delivery. In one embodiment, a hybrid mapping and ablation catheter having a planar assembly with a plurality of flexible arms and a plurality of electrodes disposed on each arm of the plurality of flexible arms, wherein at least one arm comprises: a first lumen housing a strut that extends along a length of at least one of the flexible arms; and a second lumen and a third lumen housing a conductive wire configured to independently energize one or more of the plurality of electrodes disposed on the flexible arm to deliver pulsed field ablation energy to a tissue is provided.

### CROSS-REFERENCE TO RELATED APPLICATIONS

This application claims priority to provisional application Ser. No. 63/327,908, filed Apr. 6, 2022, which is incorporated herein by reference in its entirety.

### BACKGROUND

The embodiments described herein relate generally to catheter devices for therapeutic electrical energy delivery. In particular, hybrid catheters that can be used for high powered therapeutic tissue ablation and mapping are described.

Pulsed field ablation (PFA) utilizes a controlled electric field to ablate and scar tissue through a process called irreversible electroporation (IRE). PFA provides for higher tissue specificity than conventional ablation, and its requirements for power (current 10's of Amps and voltage levels from 100's to 1000's) are higher/larger than the power requirements of high-density mapping catheters. For ablation to be successful, detailed mapping and navigation of catheters that deliver energy to specific targets are necessary

### SUMMARY

Embodiments of the present disclosure describe hybrid catheters having flexible array of electrodes configured for both mapping and high power ablation delivery. For example, catheters configured or adapted for withstanding voltage stress associated with high power ablation delivery are provided.

Accordingly, a first aspect of the present disclosure features a hybrid mapping and ablation catheter comprising: an elongate, deformable shaft comprising a proximal end and a distal end; a distal tip assembly coupled to the deformable shaft and extending distally therefrom; wherein the distal tip assembly is a planar assembly having a plurality of flexible arms and a plurality of electrodes disposed on each arm of the plurality, wherein at least one arm comprises: a first lumen housing a strut that extends along a length of at least one of the flexible arms; and a second lumen and a third lumen housing a conductive wire configured to independently energize one or more of the plurality of electrodes disposed on the flexible arm to deliver pulsed field ablation energy to a tissue. The first lumen can be centrally positioned in the arm and the second and third lumen are diametrically opposed. The catheter can further include a connector to couple the distal tip assembly to the deformable shaft, wherein an adhesive is disposed around the proximal end of the plurality of flexible arms and the connector. In some cases, the catheter further includes an adhesive disposed around a distal portion of the flexible arm comprising the first, second, and third lumen. Optionally, the catheter can further include a dielectric layer on at least one of the flexible arms, wherein a portion of the plurality of electrodes is disposed over the dielectric layer. The dielectric layer can include Parylene. Optionally, the catheter further includes a pull ring positioned proximal to the distal tip assembly, wherein the deformable shaft comprises a distal deflectable section, first and second pull wires extending along the deformable shaft, an actuator operatively coupled to a proximal end of the first and second pull wires and adapted selectively deflect the distal deflectable section, a distal end of the first and second pull wires extending through the pull ring and anchored to a distal end of the deformable shaft, wherein the pull ring is electrically isolated from the electrodes mounted on the distal tip assembly. The deformable shaft may further include a plurality of conductive wires electrically connected to the plurality of electrodes, the conductive wires extending along the deformable shaft and through the pull ring, wherein the pull wires and the conductive wires are encapsulated by tube-like material within the distal deflectable section to electrically isolate the pull ring. The distal deflectable section may define a first and a second pair of diametrically opposed lumen extending distally from the deformable shaft, wherein two lumen of the first pair house a pull wire, and at least one lumen of the second pair house a plurality of conductive wires encapsulated by the tube-like material. The tube-like material can include polyethylene terephthalate (PET). The pull ring may include a dielectric layer. In some cases, the catheter further includes a handle assembly connected to the proximal end of the deformable shaft, the handle assembly comprising an electromechanical pin-to-socket type connector, wherein the pin-to-socket type connector is configured to provide an electrical channel for each electrode mounted on the flexible arm. The electrical channels in the connector can be electrically isolated by size and positioning of the pins of the pin-to-socket type connector. The connector may further include at least one flexible circuit. Optionally, the flexible circuit forms a high-density wiring interface.

The catheter can be configured to operate within a range of about 500 Volts to at least about 2000 Volts, optionally by further comprising a connector to couple the distal tip assembly to the deformable shaft, wherein an adhesive is disposed around the proximal end of the plurality of flexible arms and the connector; an adhesive disposed around a distal portion of the flexible arm comprising the first, second and third lumen; a Parylene layer on at least one of the flexible arms, wherein the plurality of electrodes is disposed over the Parylene layer. In some cases, the catheter further includes: a pull ring positioned proximal to the distal tip assembly, wherein the deformable shaft comprises a distal deflectable section; first and second pull wires extending along the deformable shaft; an actuator operatively coupled to a proximal end of the first and second pull wires and adapted selectively deflect the distal deflectable section, wherein a distal end of the first and second pull wires extends through the pull ring and is anchored to a distal end of the deformable shaft, wherein the pull ring is electrically isolated from the electrodes mounted on the distal tip assembly. The catheter may further include conductive wires electrically isolated from the pull ring by a polyethylene terephthalate layer encapsulating the conductive wires or a dielectric layer disposed on the pull ring, and/or further include a handle assembly connected to the proximal end of the deformable shaft, the handle assembly comprising an electromechanical pin-to-socket type connector, wherein the pin-to-socket type connector is configured to provide an electrical channel for each electrode mounted on the flexible arm.

The details of one or more examples are set forth in the description below. Other features, objects, and advantages will be apparent from the description and from the claims.

### BRIEF DESCRIPTION OF DRAWINGS

Various embodiments of the present disclosure are explicated by the accompanying drawings, which are not necessarily drawn to scale. Like numerals having different letter suffixes represent different instances of substantially similar components. Reference is made to illustrative embodiments depicted in the figures, in which:

FIG. 1 is a schematic diagram view of a catheter for performing one or more diagnostic and/or therapeutic functions.

FIG. 2 is a perspective view of one embodiment of a catheter for use with the system of FIG. 1, the catheter including a catheter handle and a catheter shaft, according to one or more embodiments of the present disclosure.

FIG. 3A-C show various views of a pin-to-socket connection system for the catheter of FIG. 2.

FIG. 4 is a sectional view of a portion the catheter shaft shown in FIG. 2, showing an electrically isolated pull ring.

FIG. 5A is a plan view of a high-density electrode grid assembly of the catheter shown in FIG. 1.

FIG. 5B a perspective view of the connector of the high-density electrode grid assembly shown in FIG. 5A.

FIG. 5C are plan views of the distal end of the high-density electrode grid assembly shown in FIG. 5A, according to one or more embodiments of the present disclosure.

FIG. 6A-F are cross-sectional views of embodiments of an arm of the high-density electrode grid assembly of the catheter shown in FIG. 5C at line 6. A and B show one triple-lumen configuration with and without wires, respectively. C and D show another triple-lumen configuration with and without the arm understructure and wires, respectively. E and F show another triple-lumen configuration with and without arm understructure and wires, respectively.

### DETAILED DESCRIPTION

Embodiments of the present disclosure describe a hybrid mapping and ablation catheter.

The electrode assemblies described herein include various arrangements of electrodes. The electrodes may be activated independently from one another (in an “unganged” configuration) or may be activated together as a larger effective electrode (in a “ganged” configuration). For example, activated independent from one another, the electrodes may be used for mapping applications and/or EGM applications. In contrast, multiple electrodes may be activated together to function as a composite electrode for ablation applications and/or near field impedance navigation applications. Further, different subsets of electrode may be selectively activated (relative to one another) to provide improved control over ablation procedures.

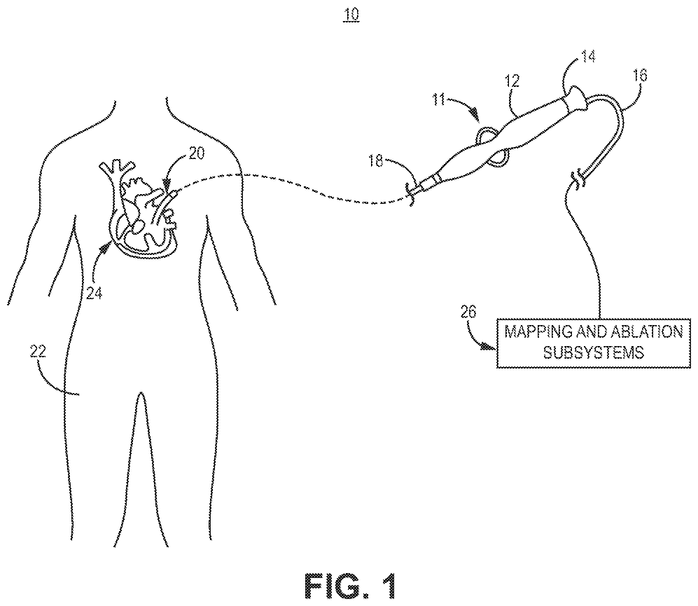

Referring to the drawings, FIG. 1 illustrates an exemplary embodiment of electrophysiological system 10 for performing one or more diagnostic and/or therapeutic functions that include components for monitoring the temperature of an electrode before, during, and/or after an ablation procedure performed on tissue 24 of a patient 22, as well as monitoring the force of contact between the electrode and tissue before, during, and/or after the ablation procedure. In the illustrative embodiment, tissue 24 is heart or cardiac tissue; however, system 10 is applicable to ablation procedures on other body tissues as well.

System 10 includes subsystems 26, which include an ablation generator and control system for communicating with and/or controlling one or more components of system 10 and mapping systems. Subsystems 26 can further include one or more of navigation systems, imaging systems and any other system or sub-system configured to perform the examination, diagnostic and/or therapeutic functions of system 10 (e.g., a positioning, recording, stimulation, and/or visualization system).

Hybrid mapping and ablation catheter 11 is part of electrophysiological system 10. Catheter 11 includes an elongate shaft 18 attached to a control handle 12 and configured for movement within the body of patient 22. Catheter 11 can be made steerable, for example by incorporating an actuator into handle 12 that is coupled to one or more steering wires that extend through elongate catheter shaft 18 and that terminate in one or more pull rings within distal region 20. Catheter 11 can be an irrigated catheter, configured to be coupled to a suitable supply of irrigation fluid and/or an irrigation pump, and/or be equipped with force feedback capabilities. As far as such features are not necessary to an understanding of the instant disclosure, they are neither illustrated in the drawings nor explained in detail herein. By way of example only, however, catheter 11 can incorporate various aspects and features of the following catheters, all from Abbott Laboratories: the FlexAbility™ ablation catheter, Sensor Enabled™; Advisor™ HG Grid Mapping Catheter, Sensor Enabled™; TactiCath™ Quartz Contact Force Ablation Catheter, Sensor Enabled™; TactiFlex™ Ablation Catheter, Sensor Enabled™.

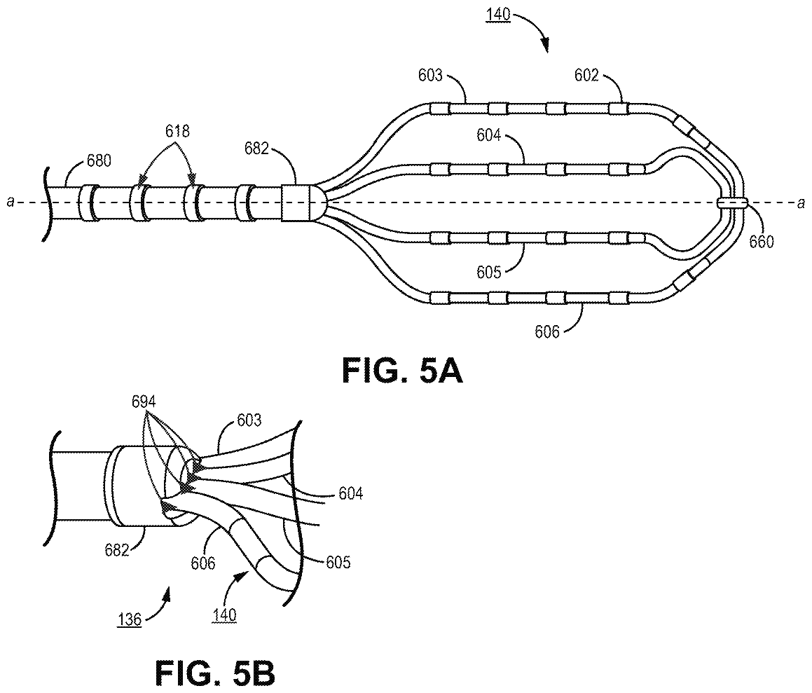

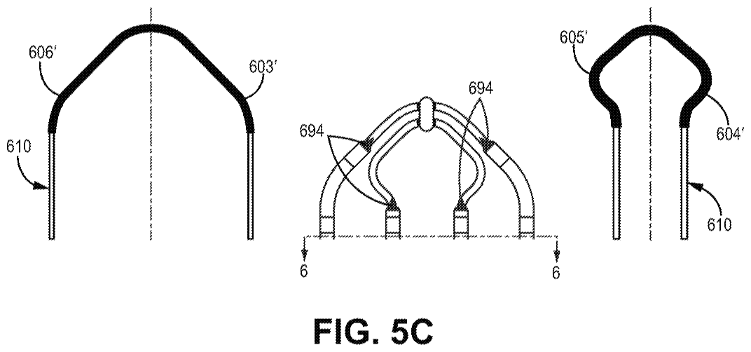

Catheter 11 further includes a plurality of electrodes (not shown) mounted in or on the distal portion 40 of the elongate shaft 18 (see FIG. 5A). The electrodes can be used, for example, in operation with a positioning, ablation, recording, stimulation, visualization, navigation, and/or mapping system (e.g., subsystems 26). The electrodes can be configured to provide a signal indicative of both a position and orientation of at least a portion of the elongate shaft 18. Handle 12 can provide mechanical and electrical connection via connector 14 for a cable 16 extending to subsystems 26. Connector 14 provides mechanical and electrical connection(s) for the one or more wires extending, for example, from subsystems 26 to one or more electrodes or sensors mounted on catheter 11. In other embodiments, connector 14 may also provide mechanical, electrical, and/or fluid connections for wires extending from other components in system 10.

Handle 12 provides a location for a clinician to hold the catheter 11 and can further provide means for steering or guiding the elongate shaft 18 within the body or tissue thereof (e.g., tissue 24) as known in the art. Catheter handles are generally conventional in the art and it will be understood that the construction of the handle 12 can vary.

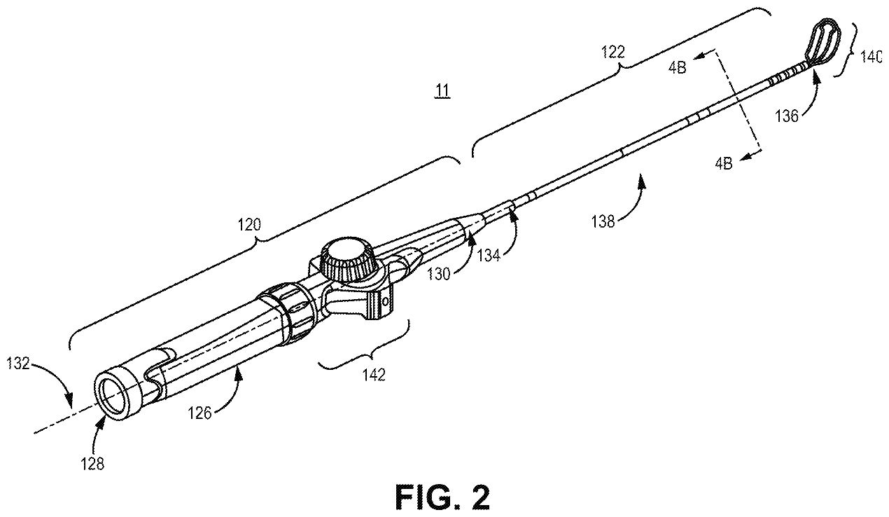

FIG. 2 illustrates one exemplary embodiment of a catheter 11 suitable for use with system 10 shown in FIG. 1. In FIG. 2, catheter 11 includes an elongated, flexible, generally cylindrical hollow shaft 22 and an ergonomically shaped control handle 120. In some embodiments, catheter 11 may include additional components such as, for example and without limitation, steering wires and actuators, irrigation lumens and ports, pressure sensors, contact sensors, temperature sensors, additional electrodes and corresponding conductors or leads, and/or ablation elements (e.g., ablation electrodes, and the like).

As shown in FIG. 2, control handle 120 includes a housing 126 extending from a proximal end 128 to a distal end 130 along a longitudinal axis 132. Housing 126 define an internal cavity that extends longitudinally. In some embodiments, a fluid lumen (not shown) may be positioned in the internal cavity (e.g., for irrigated configurations). Shaft 122 has a proximal end 134 coupled to control handle 120 and a distal end 140. In some embodiments, distal end 136 of catheter shaft 122 is deflectable, and control handle 120 is configured to control deflection of deflectable distal end 136.

While a variety of materials can be used to construct catheter shaft 122, it is typically constructed of electrically non-conductive material. Catheter shaft 122 serves as at least a portion of the blood contacting segment of the catheter 11 and is vascularly inserted into a patient by methods and means well known in the art. Catheter shaft 122 includes an elongate body 138 extending from proximal end 134 to distal end 136. In addition, elongate body 138 defines at least one lumen (not shown) extending from proximal end 134 to distal end 136. A distal tip electrode assembly 140 (shown in FIG. 5A) extends from distal end 136 of catheter shaft 122. Distal tip electrode assembly 140 includes a plurality of electrodes (shown in FIG. 5A), which may be used for a variety of diagnostic and therapeutic purposes including, for example and without limitation, cardiac mapping and/or ablation.

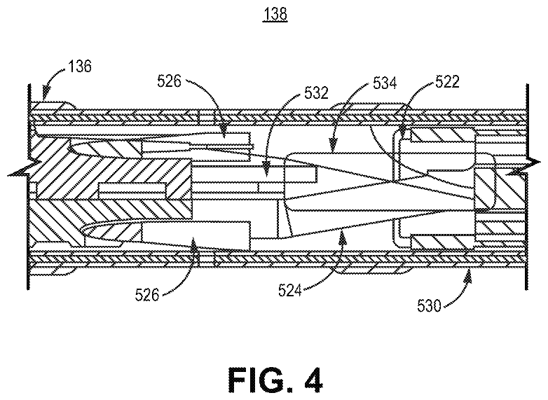

In at least some embodiments where the distal end 136 is deflectable, control handle 120 includes at least one actuator 142 to allow an operator to adjust selectively deflect distal portion 136 of catheter shaft 122. The selective adjustment may be achieved through the use of one or more pull wires positioned within a lumen of catheter 2 (e.g., see FIG. 4). The pull wires extend through elongate body 138 and terminate in one or more pull rings within the distal shaft.

Catheter shaft 122 includes a plurality of lumens defined by elongate body 138 and extending from proximal end 134 to distal end 136. For example, distal end 136 of catheter shaft 122 can include four lumens (not shown), through which electrical conductor wires and/or actuation wires can extend. Electrical conductor wires generally extend from the control handle to the distal end. Electrical conductor and/or actuation wires can be any of the electrical conductor or actuation wire types known in the art including, for example and without limitation, the types described in US2021/0128230A1 and US2015/0119859A1, which are incorporated by reference herein. The lumen carrying electrical conductor wires can be partially or completely surrounded by or encapsulated by an insulative material.

In addition, or alternatively, insulative material can be provided in other configurations, such as provided by a tube-like configuration. The tubing can be coterminous with the electrical conductor wire (i.e., the tubing length is not limited to the length of the catheter shaft). Although conventional catheters include polyimide tubing, the insulative material for the electrical conductor wires of a hybrid mapping and ablation catheter of the present disclosure can include high-density polyethylene, polyaryletherketones, polyetheretherketones, polyurethane, polypropylene, oriented polypropylene, polyethylene, crystallized polyethylene terephthalate, polyethylene terephthalate, polyester, polyetherimide, acetyl, ceramics, and various combinations thereof to increase electrical insulation around conductor wires.

A wire connector, such as connector 14 shown in FIG. 1, may be positioned at proximal end 128 of control handle 120. Each electrode of distal tip electrode assembly 140 may be connected to an electrical conductor wire that extends to the wire connector through catheter shaft 122 and control handle 120. The wire connector is adapted to be connected to a device of one or more of subsystems 26.

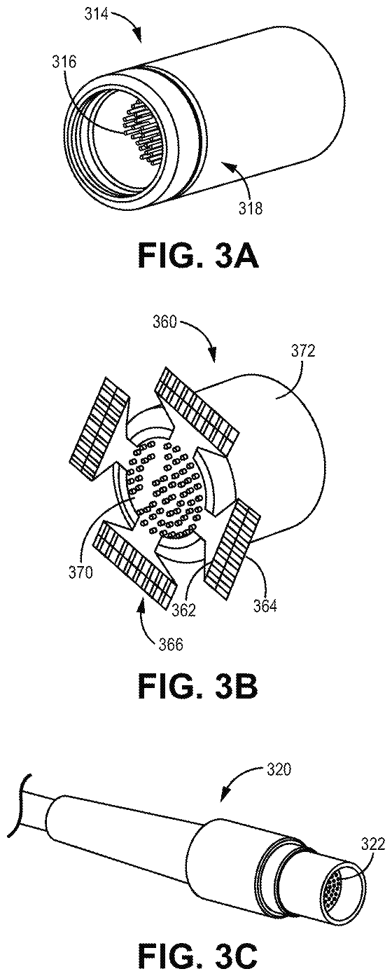

Turning to FIGS. 3A-3C, electromechanical pin-to-socket type connectors can be used for connector 14. FIGS. 3A and 3B illustrate two embodiments of male connectors for placement on proximal end 128 of FIG. 2. FIG. 3C depicts an exemplary female connector 320 of subsystems 26 configured for electrical connection with a male connector. In some cases, connector 14 is a pin-type connector such as male connector 314 having a plurality of pins 316 and engagement shaft 318 configured to mate with a corresponding portion of a socket-type connector, such as female connector 320 having a plurality of sockets 322. Each pin and socket is electrically and mechanically connected or coupled to provide an electrical channel between catheter 2 and subsystems 26. The pins 316 of the male connector 314 and the sockets 322 of the female connector 320 are configured to correspond with each other when the connectors are joined. The male and female connectors may be keyed to ensure proper orientation. Engagement of the connectors can be enhanced with a spring force, pressure force, mechanical connection or other means to ensure contact between the pins and sockets.

The number and type of pins 316 are configured to allow all electrodes on the distal tip assembly to be electrically connected during a PFA procedure. For example, where the distal tip is a high-density electrode assembly comprising an array of 18 or more electrodes, male connector 314 can include a pin for each electrode. The connector is preferably constructed so that liquid spillage in normal use does not wet electrical or other components. In another embodiment, connector 14 may include sockets instead of pins, and may connect to a matching pin connector.

Isolation between the data channels in the connector can be accomplished through sizing and positioning of the pins and/or size and spacing of conductors corresponding to nodes on a flexible circuit. The number of data channels carried by the connector can be dependent on the size of the pins in the connector and the overall size of the connector itself. Pins must also be rigid enough to maintain adequate performance of the connector which sets a lower limit on the size of the pins. Thus, in order to increase the number of data channels in pin-to-socket connectors, the size of the connector must increase to accommodate the increase in pins. This often requires an increase in the size of the handle in a medical device at its proximal end, which is not always desirable.

FIG. 3B shows a connector including flexible circuits and adapted for connection to a conventional socket connector, e.g., the female connector 320. The flexible circuits allow for a higher capacity of data channels while providing easier assembly. For example, the number of data channels carried by the connector is in part dependent on the size/spacing of the conductors corresponding to nodes of the flexible circuit. Flexible circuit connector 360 comprises flexible circuit 366 having a base area 362 and tab areas 364. Each tab 364 includes a flexible circuit 366 forming a high-density wiring interface. A plurality of pins are electrically connected to each of the contact nodes 370 on the opposite side (not shown) of the base area 362 of the flexible circuit 366. A cover 372 surrounds the pins and may provide mechanical means for connecting to a matching socket connector. In another embodiment, flexible circuit connector 360 may include sockets instead of pins and may connect to a matching pin connector. In either embodiment, the matching connector may be a conventional socket or pin connector, respectively.

Referring to FIG. 4, elongate body 138 can include a pull ring 522 that interfaces with distal shaft coupler 530. Pull wires 532 (e.g., actuation wires) can be connected to the electrode assembly through pull ring 522. Upon actuation of the pull wires, catheter shaft 122 can be deflected in some embodiments. A plurality of conductive wires (not shown) and/or pull wires 523 can extend through a center of the pull ring 522. In an example, the conductive wires can be electrically coupled with one or more sensors and/or electrodes. The pull wires are anchored distally from pull ring 522. Conductive wires and each pull wire extend through pull ring 522. Pull wires 524 are secured in the distal shaft by anchors 526. The conductive wires and/or pull wires extending the distance between the distal end and pull ring 522 are encapsulated by tube-like material to electrically isolate pull ring 522. For example, the conductive wires and pull wires may be encapsulated by tube 534 made of a chemically inert, biologically stable, and biocompatible material such as polyetherimide, polyimide, PTFE or other high-temperature polymers. In some embodiments, pull ring 522 includes a dielectric layer for electrical isolation. Exemplary dielectric layer material includes chemically inert, biologically stable, and biocompatible materials, such as, but not limited to, polymers such as Parylene, polyimide, polyetheretherketone (PEEK), polyethylene terephthalate (PET), polytetrafluoroethylene (PTFE) (e.g., Teflon), polyether block amides (e.g., PEBAX), polyurethane, epoxy resins (e.g., SU-8), and blends thereof. The layer can be applied as a coating in addition to or in lieu of encapsulation of the conductive wires or pull wires. Pull ring 522 may be partially coated. For example, the interior of pull ring 522 can include a layer of dielectric material, e.g., polyetherimide, polyimide, PTFE or other high-temperature polymers, to electrically isolate the pull ring.

FIG. 5A-C illustrate embodiments of distal tip electrode assembly 140, which include a plurality of electrodes mounted or otherwise attached to an electrode support member configured as a planar array. Distal tip electrode assembly 140 is shaped to facilitate maneuverability in and/or contact with certain anatomical structures. The electrode support member can include an understructure formed of any flexible, superelastic, pseudoelastic, or semi-rigid (e.g., deformable) material capable of facilitating sufficient maneuverability within patient's tissue for efficient mapping and/or ablation. Suitable materials include, for example, metal alloys, stainless steel (including spring stainless steel sold under the trade name Elgiloy® and Hastelloy®), copper-aluminum-nickel alloys, CoCrNi alloys (e.g., sold under the trade name Phynox), MP35N®, CoCrMo alloys, alloys including zinc, copper, gold, and/or iron, polymers including any of the above materials, shape memory polymers, and/or combinations thereof. In some cases, the electrode support member is constructed entirely, or in part, of nitinol. Nitinol is an approximately stoichiometric alloy of nickel and titanium, which may also include minor amounts of other metals to achieve desired properties. The electrode support member can have any length, shape or configuration suitable or necessary for a desired therapy. In some cases, an electrode support member has a rectangular, square, circular, or elliptical cross-section, or a cross-section that varies along the length of the electrode support member.

Distal tip electrode assembly 140 can be adapted to conform to tissue (e.g., cardiac tissue). For example, distal tip electrode assembly 140 can deflect, allowing the flexible framework to conform to the tissue. In some embodiments, the construction (including, for example, the length and/or diameter of the arms) and material of the arms can be adjusted or tailored to be created, for example, desired resiliency, flexibility, foldability, conformability, and stiffness characteristics, including one or more characteristics that may vary from the proximal end of a single arm to the distal end of that arm, or between or among the plurality of arms comprising a single paddle structure. The foldability of materials such as nitinol and/or flexible substrate provide the additional advantage of facilitating insertion of the planar structure into a delivery catheter or introducer, whether during delivery of the catheter into the body or removal of the catheter from the body at the end of a procedure.

The high-density planar array (or ‘paddle’ configuration) of electrodes 602 shown in FIG. 5A comprises four side-by-side, longitudinally-extending arms 603, 604, 605, 606, which can form a flexible framework on which the electrodes 602 are disposed. Arms 603-606 are joined by coupler 660, which forms the distalmost point of catheter 11. The arms are laterally separated from each other. Each of the four arms carries a plurality of electrodes 602, which can be spaced along a length of each of the four arms. Electrodes 602 are illustrated as ring electrodes, however, split ring, ring segment (or other durable electrode), flexible and/or printed electrodes, or a combination thereof, can be used in place of any ring electrode. In addition, one or more of arms 603-606 can include one or more flexible circuits and/or printed conductive traces. Although FIG. 5A depicts four arms, distal tip assembly 140 could be configured with more or fewer arms. Additionally, while 18 electrodes are shown, (e.g., five electrodes on first outboard arm 603 and second outboard arm 606 and four electrodes on first inboard arm 604 and second inboard arm 605), the catheters can include more or fewer than 18 electrodes. In addition, the first outboard arm 603 and second outboard arm 606 can include more or fewer than five electrodes and the first inboard arm 604 and second inboard arm 605 can include more or fewer than 4 electrodes).

In some embodiments, the electrodes 602 can be used in diagnostic, therapeutic, and/or mapping procedures, such as for electrophysiological studies, pacing, cardiac mapping, and ablation. In some embodiments, the electrodes 602 can be used to perform unipolar or bipolar ablation. This unipolar or bipolar ablation can create specific lines or patterns of lesions. In some embodiments, the electrodes 602 can receive electrical signals from the heart, which can be used for electrophysiological studies. In some embodiments, the electrodes 602 can perform a location or position sensing function related to cardiac mapping. In some embodiments, catheter 10 can include a catheter shaft 122. As depicted, a proximal portion 680 is disposed in the distal end of the catheter shaft 122, and mounted with shaft electrodes 618. In a non-limiting example, shaft electrodes are ring electrode or ring-segment electrodes; other electrodes may be used, based on the desired application. Shaft electrodes 618 can be configured for diagnostic, therapeutic, and/or mapping procedures. Although four shaft electrodes 618 are illustrated, embodiments with fewer or more than four electrodes are within the scope of this disclosure. A connector 682, disposed at the distal end of the catheter shaft 122, is configured to hold the four longitudinally-extending arms 603, 604, 605 and 606 in plane. In some cases, the electrodes 602 are disposed directly on the understructure that forms each one of the arms 603, 604, 605 and 606, which understructure may be constructed of a flexible material, metal, or alloy thereof. In some embodiments, the understructure is inserted in a tubing, such as a non-conductive and/or heat shrink tubing, which extends from the distal end of connector 682. The electrodes 602 can then be mounted, applied or otherwise disposed on the exterior of the tubing.

In some embodiments, at least some of the arms 603, 604, 605 and 606 may be electrically conductive and selectively activatable as electrodes. Further, in some embodiments, for electrophysiology measurements, at least some of electrodes 602 and arms 603, 604, 605 and 606 may be activated independent from one another in an “unganged” configuration (i.e., may sense voltages independent from one another, be energized independent from one another, may be energized at different polarities from one another, and/or may be energized at different voltages from one another). In contrast, for ablation applications, two or more of electrodes 602 and arms 603, 604, 605 and 606 may be activated in unison in a “ganged’ configuration to form a larger effective electrode. Those of skill in the art will appreciate that any suitable combination of electrodes may be activated in unison.

In some embodiments, an adhesive 694 (illustrated as solid triangles) can be disposed around the proximal end of the transitional arms and the connector 682. Non-limiting examples of suitable adhesive include biocompatible epoxy, or the like. Adhesive 694 can also be disposed slightly distally of the distalmost electrode 602 and/or at coupler 660. For example, the embodiment shown in the center view of FIG. 5C has adhesive 694 disposed on each arm of the high-density planar array at a distal transitional edge of an outer tubing (e.g., heat-shrink and/or non-conductive tubing, as discussed above). The adhesive 694 is disposed at the chamfer between the tubing diameter and the arm.

Also shown in FIG. 5C, in one embodiment of a distal tip assembly wherein the understructure of arms 603′, 604′, 605′, and 606′ is constructed of nitinol, the arms are at least partially covered with dielectric layer 610. For example, surfaces of the arms that have electrodes mounted thereon may be coated or otherwise covered with a dielectric material. Dielectric layer 610 may be formed of a chemically inert, biologically stable, and biocompatible material with high dielectric properties. In some cases, the material can be applied in a very thin layer. An exemplary dielectric layer may include thin coats of polyester, polyamides, polyimides, and blends of polyurethane and polyimides. In one embodiment, for example only and without limitation, dielectric layer 610 may be Parylene (e.g., Parylene C, Parylene N) or an acrylated urethane. In various embodiments, the thickness of the dielectric layer 610 may range from about 0.0001 mm to about 0.05 mm, or from about 0.0003 mm to about 0.0006 mm. The dielectric layer can be applied to the arms by spray-coating, dip-coating, or other chemical deposition method known in the art. Alternatively, dielectric layer 610 can be achieved by placing a heat shrink tube or polymer tube on the nitinol understructure. Exemplary heat shrink or polymer tube material can include fluorinated ethylene-propylene copolymer (FEP), polytetrafluoroethylene (PTFE), polyethylene terephthalate (PET), or the like. In some embodiments where the electrode support member is an electrically conductive material, dielectric layer 610 can electrically insulate electrodes and/or conductive traces from the electrically conductive material.

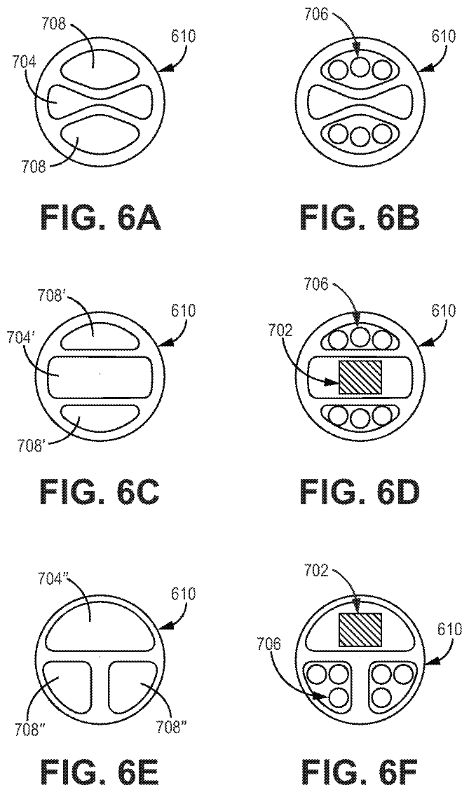

FIGS. 6A-6F show a cross-sectional view of various triple-lumen embodiments of any one of arms 603′-606′ at line 6-6 of FIG. 5C. FIGS. 6B, 6D, and 6F each illustrate a non-limiting embodiment for packing of the lumens shown in FIGS. 6A, 6C, and 6E, respectively. An arm understructure 702 (e.g., strut) can be housed in major lumen 704 (shown as 704′ and 704″) and smaller lumen 708, 708′, and 708″ can house wires 706 for sensors, interactive elements, and/or fluid. The triple-lumen cross-section can possess bilateral symmetry (e.g., FIGS. 6A and 6B) or the lumen can be arranged asymmetrically (FIG. 6C). The lumen can be defined by a layer of dielectric material disposed about the understructure 702. The dielectric material can be any electrically-insulating, material that provides electrode to electrode isolation along the strut. As shown, the layer of dielectric material is the same material as dielectric layer 610. Various energization patterns of electrodes can be selectively implemented to produce a desired lesion surface area, volume, and/or depth, including as areas of continuous or non-continuous lesions, with regularly spaced gaps of untreated tissue. Electrodes mounted on the surface of the arms may be configured to facilitate measurement of electrical characteristics of tissue, independently.

Advantages of a catheter of the present disclosure include, but are not limited to, robust, yet maneuverable configurations to facilitate high-density mapping of electrophysiological activity in hard-to-reach areas, before and after ablation, without compromising the speed with which maps can be generated, or the efficiency of therapy delivery. The high power ablation and efficient remapping capabilities of the disclosed catheters may improve procedure outcome. For example, embodiments of the present disclosure allow for tissue specific lesion formation within a shortened procedure time.

The preceding examples are intended to illustrate the above invention and should not be construed as to narrow its scope. The scope of this disclosure should be determined by the appended claims and their legal equivalents. Variations and modifications may be made while remaining within the scope of one or more embodiments of the present disclosure. Various features and aspects of the disclosed embodiments can be combined with or substituted for one another in order to form various embodiments.

The terms “proximal” and “distal” are be used throughout the specification with reference to a clinician manipulating one end of an instrument used to treat a patient. Thus, the term “proximal” refers to the portion of the instrument closest to the clinician and the term “distal” refers to the portion located furthest from the clinician. Surgical instruments may be used in many orientations and positions, however, and these terms are not intended to be limiting and absolute.

Patent literature, or other disclosure material, which is said to be incorporated by reference herein, in whole or in part, is incorporated to the extent that the incorporated materials do not conflict with existing definitions, statements, or other disclosures set forth in the present disclosure.

What is claimed is:

1. A hybrid mapping and ablation catheter comprising:
an elongate, deformable shaft comprising a proximal end and a distal end;
a distal tip assembly extending distally from the distal end of the deformable shaft;
wherein the distal tip assembly is a planar assembly having a plurality of flexible arms and a plurality of electrodes disposed on each arm of the plurality of flexible arms, wherein at least one arm comprises:
a first lumen housing a strut that extends along a length of at least one of the flexible arms; and
a second lumen and a third lumen housing a conductive wire configured to independently energize one or more of the plurality of electrodes disposed on the flexible arm to deliver pulsed field ablation energy to a tissue.

2. The catheter of claim 1, wherein the first lumen is centrally positioned in the arm and the second and third lumen are diametrically opposed.

3. The catheter of claim 1, further comprising a connector to couple the distal tip assembly to the deformable shaft, wherein an adhesive is disposed around the proximal end of the plurality of flexible arms.

4. The catheter of claim 3, further comprising an adhesive disposed around a distal portion of the flexible arm comprising the first, second, and third lumen.

5. The catheter of claim 1, further comprising a dielectric layer on at least one of the flexible arms, wherein a portion of the plurality of electrodes is disposed over the dielectric layer.

6. The catheter of claim 5, wherein the dielectric layer comprises Parylene.

7. The catheter of claim 1, further comprising a pull ring positioned proximal to the distal tip assembly, wherein the deformable shaft comprises a distal deflectable section, first and second pull wires extending along the deformable shaft, an actuator operatively coupled to a proximal end of the first and second pull wires and adapted selectively deflect the distal deflectable section, a distal end of the first and second pull wires extending through the pull ring and anchored to a distal end of the deformable shaft, wherein the pull ring is electrically isolated from the electrodes mounted on the distal tip assembly.

8. The catheter of claim 7, wherein the deformable shaft further comprises a plurality of conductive wires electrically connected to the plurality of electrodes, the conductive wires extending along the deformable shaft and through the pull ring, wherein the pull wires and the conductive wires are encapsulated by tube-like material within the distal deflectable section to electrically isolate the pull ring.

9. The catheter of claim 8, wherein the distal deflectable section defines a first and a second pair of diametrically opposed lumens extending distally from the deformable shaft, wherein two lumens of the first pair house a pull wire, and at least one lumen of the second pair houses a plurality of conductive wires encapsulated by the tube-like material.

10. The catheter of claim 9, wherein the tube-like material comprises polyethylene terephthalate.

11. The catheter of claim 7, wherein the pull ring comprises a dielectric layer.

12. The catheter of claim 1, further comprising a handle assembly connected to the proximal end of the deformable shaft, the handle assembly comprising an electromechanical pin-to-socket connector, wherein the pin-to-socket connector is configured to provide an electrical channel for each electrode mounted on the flexible arm.

13. The catheter of claim 12, wherein the electrical channels in the connector are electrically isolated by size and positioning of the pins of the pin-to-socket connector.

14. The catheter of claim 12, wherein the connector further comprises at least one flexible circuit.

15. The catheter of claim 14, wherein the flexible circuit forms a high-density wiring interface.

16. The catheter of claim 1 configured to operate within a range of about 500 Volts to at least about 2000 Volts.

17. The catheter of claim 16, further comprising:
a connector to couple the distal tip assembly to the deformable shaft, wherein an adhesive is disposed around the proximal end of the plurality of flexible arms and the connector;
an adhesive disposed around a distal portion of the flexible arm comprising the first, second and third lumen;
a Parylene layer on at least one of the flexible arms, wherein the plurality of electrodes is disposed over the Parylene layer.

18. The catheter of claim 17, further comprising:
a pull ring positioned proximal to the distal tip assembly, wherein the deformable shaft comprises a distal deflectable section;
first and second pull wires extending along the deformable shaft;
an actuator operatively coupled to a proximal end of the first and second pull wires and adapted selectively deflect the distal deflectable section
wherein a distal end of the first and second pull wires extends through the pull ring and is anchored to a distal end of the deformable shaft,
wherein the pull ring is electrically isolated from the electrodes mounted on the distal tip assembly.

19. The catheter of claim 18, further comprising conductive wires electrically isolated from the pull ring by a polyethylene terephthalate layer encapsulating the conductive wires or a dielectric layer disposed on the pull ring.

20. The catheter of claim 19, further comprising a handle assembly connected to the proximal end of the deformable shaft, the handle assembly comprising an electromechanical pin-to-socket type connector, wherein the pin-to-socket type connector is configured to provide an electrical channel for each electrode mounted on the flexible arm.

21. The catheter of claim 1, wherein at least some of the plurality of electrodes are configured to be activated independent from one another in an unganged configuration.

22. The catheter of claim 1, wherein at least some of the plurality of electrodes are configured to be activated in unison in a ganged configuration.

23. The catheter of claim 1, wherein each arm is electrically conductive and is configured to operate as an electrode.

24. The catheter of claim 23, wherein at least some electrodes in a group of electrodes including the plurality of electrodes and each arm are configured to be activated independent from one another in an unganged configuration.

25. The catheter of claim 23, wherein at least some electrodes in a group of electrodes including the plurality of electrodes and each arm are configured to be activated in unison in a ganged configuration.
